(19) United States
(12) Patent Application Publication
(10) Pub. No.: US 2025/0325452 A1
(43) Pub. Date: Oct. 23, 2025

(54) OAT PROTEIN SPONGE FILTER FOR FEEDING BOTTLES AND METHOD OF PREPARATION THEREOF

(71) Applicant: PES University, Bengaluru (IN)

(72) Inventors: ANANYA M, Bangalore (IN); Adithi G R, Bangalore (IN); Nidhi N Gambhir, Bangalore (IN)

(21) Appl. No.: 19/034,581

(22) Filed: Jan. 23, 2025

(30) Foreign Application Priority Data
Apr. 18, 2024 (IN) 202441031262

(51) Int. Cl. A61J 9/08; A23C 7/04; B01D 39/16; C07K 1/14; C07K 14/415

(52) U.S. Cl. CPC A61J 9/08; A23C 7/04; B01D 39/1692; C07K 1/14; C07K 14/415

(57) ABSTRACT

The present disclosure discloses a method for preparing an Oat Protein Sponge (OPS) filter for feeding bottles to remove microplastics. The method involves isolating proteins from oats using cell disruption with alkali. The oats are cultivated through tissue culture and/or hydroponics. The isolated protein is then made soluble through incubation and breakdown, followed by neutralization with a strong acid such as Hydrochloric or Sulfuric acid. The resulting protein is concentrated oat protein, treated with a crosslinking agent, and formed into a sponge structure. This structure is incubated to create a stable hydrogel, which is subsequently washed and purified. The OPS filter effectively removes microplastics from milk and is integrated into feeding bottles, providing a solution to enhance milk safety and quality for infants.

### CROSS-REFERENCE TO RELATED APPLICATION

This application is based upon and claims priority from, Application No. IN 202441031262, filed on 18 Apr. 2024 the entireties which are incorporated herein by reference for all purposes.

### BACKGROUND

### Field of the Present Invention

The present disclosure relates to the field of biotechnology, and in particular, relates to the development of bio-based filtration material to remove microplastic contaminants from milk.

### Background of the Related Art

Microplastics, plastic particles smaller than 5 millimeters, have become a global environmental and health concern. These particles originate from the breakdown of larger plastics or are intentionally added to products. These include the direct release from personal care products, synthetic clothing fibers shedding during washing, and degradation of larger plastic items like bottles and packaging. Such microplastics persist for long periods due to their resistance to biodegradation and accumulate in water bodies, soils, and even the air. Further, microplastic ingestion can lead to physical harm, blockages in digestive systems, and toxicological effects due to chemicals that may adhere to or leach from the plastics. Furthermore, the microplastics act as vectors for transporting pollutants and pathogens, which adds complexity to their ecological impact. Additionally, the microplastics lead to severe human health risks, especially related to food and water contamination. Additionally, the microplastics are known to be present in both cow's milk and breast milk which is a big concern about infant health, as their developing systems are more vulnerable in comparison to adults.

Existing methods for removing microplastics from milk include centrifugation, filtration, and adsorption. While centrifugation can separate larger microplastics, it often fails with smaller particles and is costly for smaller producers. Further, the filtration techniques, like ultrafiltration (UF) and nanofiltration (NF), face issues with clogging and nutrient loss. Other filtration techniques such as Reverse Osmosis (RO), are highly effective but expensive and can strip essential nutrients from the milk, requiring additional enrichment. Additionally, biological filtration is another emerging approach, utilizing chitosan, derived from crustacean shells, to bind and remove microplastics. However, such chitosan-based filtration lacks long-term effectiveness and there are associated safety issues in milk processing because there are concerns about potential allergens or other unwanted compounds being introduced into the milk through such chitosan-based filters.

Furthermore, the adsorption methods, such as using activated carbon or magnetic nanoparticles, are efficient method for filtration of liquids but cannot be used for filtration of the milk as it will remove essential nutrients from the milk necessary for the growth of the infant. Apart from this, other adsorption-based techniques, such as those using metal-organic frameworks (MOFs), aerogels, and sponges, have shown potential for removing microplastics and nano-plastics from water bodies. Although these materials offer practical reusability and rapid adsorption capabilities, they also cannot be used for filtration of the milk as they will also remove essential nutrients from the milk and/or introduce new contaminants, thereby negatively impacting milk quality.

Additionally, it is important to note that the current filtration techniques primarily utilize biodegradable materials designed for applications outside of milk processing. These filters, although effective in other contexts, have not been specifically optimized for milk, resulting in concerns regarding their efficacy, safety, and impact on nutritional content.

Therefore, there is a need for a new and more advanced filter for feeding bottles to filter the microplastic to retain the milk quality and overcome the above-mentioned drawbacks of the existing technology.

### BRIEF SUMMARY

One or more embodiments are directed to a method of preparation of an Oat Protein Sponge (OPS) filter for a feeding bottle. In an embodiment, the method includes obtaining an oat protein matrix by isolating proteins from oats by cell disruption using an alkali. The oats are cultivated through tissue culture and/or hydroponics. In an embodiment, the alkali includes Sodium Hydroxide (NaOH) and/or Potassium Hydroxide (KOH).

In an embodiment, the method includes forming a soluble oat protein by incubating and breaking down the obtained protein matrix. In an embodiment, the method includes neutralizing the formed soluble oat protein by a strong acid. The strong acid includes Hydrochloric acid (HCl) and/or Sulfuric acid (H₂SO₄). In an embodiment, the method includes obtaining a concentrated oat protein by separating, purifying, and drying the neutralized oat protein.

In an embodiment, the method includes obtaining an oat protein sponge structure by treating the obtained concentrated oat protein with a crosslinking agent. The crosslinking agent includes epichlorohydrin. In an embodiment, treating the obtained concentrated oat protein includes adding a predefined amount of the crosslinking agent to the obtained concentrated oat protein. The predefined amount is 600 μL. Further, treating the obtained concentrated oat protein includes stirring for a predefined time interval to obtain the oat protein sponge structure. The predefined time interval is 2 hours

In an embodiment, the method includes obtaining a stable hydrogel structure by incubating the obtained oat protein sponge structure. The incubation of the obtained oat protein sponge structure to obtain the stable hydrogel structure is performed for a time interval ranging from 8 to 12 hours.

In an embodiment, the method includes washing and purifying the stable hydrogel structure with deionized water to prepare the OPS filter for a feeding bottle. The OPS filter filters out microplastics from milk stored in the feeding bottles during feeding sessions. Further, the OPS filter has a pore size range from 0.49 to 1.54 μm to remove the microplastics from the milk and allow passage of nutrients. Furthermore, the OPS filter adsorption efficiency ranges from 75% to 81.2% at a pH of 6.

One or more embodiments are directed to a feeding bottle with an Oat Protein Sponge (OPS). In an embodiment, the feeding bottle includes a container to store milk. Further, the feeding bottle includes a cap to be placed on the container to prevent the stored milk from spilling and contamination. Furthermore, the feeding bottle includes a rubber nipple coupled to the cap for egressing the milk during feeding sessions. Moreover, the feeding bottle includes an Oat Protein Filter (OPS) installed in the feeding bottle, such that the OPS filters microplastics from the stored milk before the milk egresses from the rubber nipple during the feeding sessions.

In an embodiment, the OPS is formed by the steps including obtaining an oat protein matrix by isolating proteins from oats by cell disruption using an alkali. Further, the steps include forming a soluble oat protein by incubating and breaking down the obtained protein matrix. Furthermore, the steps include neutralizing the formed soluble oat protein by a strong acid. Moreover, the steps include obtaining a concentrated oat protein by separating, purifying, and drying the neutralized oat protein. Additionally, the steps include obtaining an oat protein sponge structure by treating the obtained concentrated oat protein with a crosslinking agent. In an embodiment, the OPS is formed by the steps including obtaining a stable hydrogel structure by incubating the obtained oat protein sponge structure. Further, the steps include washing and purifying the stable hydrogel structure with deionized water to prepare the OPS filter for a feeding bottle.

The features and advantages of the subject matter here will become more apparent in light of the following detailed description of selected embodiments, as illustrated in the accompanying FIGUREs. As will be realized, the subject matter disclosed is capable of modifications in various respects, all without departing from the scope of the subject matter. Accordingly, the drawings and the description are to be regarded as illustrative in nature.

### BRIEF DESCRIPTION OF THE DRAWINGS

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates a feeding bottle with an Oat Protein Sponge (OPS) filter, in accordance with an embodiment of the present disclosure.

FIG. 2 illustrates a flowchart of method for the preparation of the Oat Protein Sponge (OPS) filter for feeding bottles, in accordance with an embodiment of the present disclosure.

Other features of embodiments of the present disclosure will be apparent from the accompanying drawings and detailed description that follows.

### DETAILED DESCRIPTION OF THE INVENTION

### Terminology

Brief definitions of terms used throughout this application are given below.

The terms “connected” or “coupled” or “attached”, and related terms are used in an operational sense and are not necessarily limited to a direct connection or coupling. Thus, for example, two devices/equipment/components may be coupled directly, or via one or more intermediary devices/equipment/components. As another example, devices/equipments/components may be coupled in such a way that information can be passed there between, while not sharing any physical connection with one another. Based on the disclosure provided herein, one of ordinary skills in the art will appreciate a variety of ways in which connection or coupling exists in accordance with the aforementioned definition.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context dictates otherwise. Also, as used in the description herein, the meaning of “in” includes “in” and “on” unless the context dictates otherwise.

The phrases “in an embodiment,” “according to one embodiment,” and the like generally mean the particular feature, structure, or characteristic following the phrase is included in at least one embodiment of the present disclosure and may be included in more than one embodiment of the present disclosure. Importantly, such phrases do not necessarily refer to the same embodiment.

Exemplary embodiments will now be described more fully hereinafter with reference to the accompanying drawings, in which exemplary embodiments are shown. This disclosure may, however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

One or more embodiments are directed to a method of the preparation of an Oat Protein Sponge (OPS) filter for feeding bottles. The OPS Filter enhances and/or improves the quality of milk by removing contaminants such as microplastics. In an embodiment, the method includes obtaining proteins from oats, which may be cultivated through tissue culture or hydroponics, and disrupting the cells using an alkali such as Sodium Hydroxide (NaOH) or Potassium Hydroxide (KOH). The proteins may be processed to form a soluble oat protein through extraction, concentration, and controlled enzymatic or physical treatment. Further, the soluble oat protein may be neutralized with a strong acid like Hydrochloric acid (HCl) or Sulfuric acid (H₂SO₄) to obtain concentrated oat protein. The concentrated oat protein may then be treated with a crosslinking agent such as epichlorohydrin to form a sponge structure, which may be further incubated to develop a stable hydrogel. Furthermore, the hydrogel may be washed and purified to create the OPS filter. The OPS filter may filter microplastics with a pore size ranging from 0.49 to 1.54 μm, achieving an adsorption efficiency of 75% to 81.2% at pH 6, and exhibiting a compressive strength of 176 kPa and a strain capacity of 66%. Furthermore, the disclosed invention includes a container for storing milk, a cap to prevent spillage and contamination, and a rubber nipple that facilitates the egress of milk during feeding sessions. The OPS filter may be positioned in the bottle to remove microplastics from the milk before it passes through the rubber nipple. Furthermore, the materials used for the bottle's container and cap may include metal, plastic, fiber, or silicon.

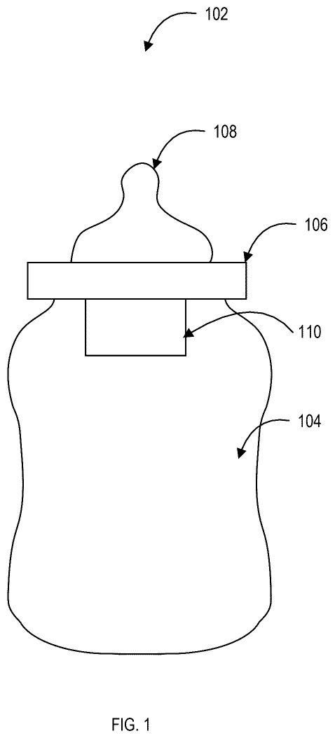

FIG. 1 illustrates a feeding bottle 102 integrated with an Oat Protein Sponge (OPS) filter 110, in accordance with one or more embodiments of the present disclosure. In an embodiment, the feeding bottle 102 includes a container 104, a cap 106, a rubber nipple 108, and the OPS filter 110. The feeding bottle 102 may be utilized to store and dispense milk. Further, the feeding bottle 102 may be utilized to store and dispense other liquids, such as water. In an embodiment, the container 104 may store the milk for consumption. Further, the container 104 may be made from materials that offer durability, resistance to chemical interactions, and structural integrity. Furthermore, the container 104 may be made from materials, such as glass, stainless steel, or medical-grade silicone. Moreover, the container 104 may be a cylindrical or conical shape, with optional graduated markings for volume measurement, enhancing usability during feeding sessions. Additionally, the container 104 may include an ergonomic design, providing a comfortable grip for caregivers.

In an embodiment, the cap 106 may be placed on the container 104 to prevent the stored milk from spilling and contamination. Further, the cap 106 may be made of materials suitable for maintaining hygiene and withstanding sterilization processes, such as plastic or silicone. Furthermore, the cap 106 may incorporate a built-in pressure-regulating mechanism or venting valve to prevent the formation of a vacuum within the container 104 during feeding sessions, ensuring a consistent flow of milk.

In an embodiment, the rubber nipple 108 may be coupled to the cap 106, for egressing the milk during feeding sessions. The rubber nipple 108 may simulate the feel of natural breastfeeding and provide an infant with a comfortable and natural feeding experience. Further, the rubber nipple 108 may include flow-rate control features to facilitate the milk flow tailored to the infant feeding needs. In an embodiment, the rubber nipple 108 may include anti-colic designs, such as vents or valves, to reduce air intake and mitigate digestive discomfort.

In an embodiment, a latex nipple may be coupled to the cap 106, for egressing the milk. The latex nipple may be an alternative to the rubber nipple 108 and may provide similar benefits in terms of comfort and flow-rate control. The latex nipple may simulate the feel of natural breastfeeding and offer varying flow rates to accommodate different infant feeding stages. Further, the latex nipple may include one or more anti-colic features, such as strategically placed air vents or one-way valves, that are configured to allow air to escape from the bottle during feeding.

In an embodiment, the OPS filter 110 may be installed within the feeding bottle 102. The OPS filter 110 may filter microplastics from the stored milk before the milk egresses from the rubber nipple 108 during the feeding session.

In an embodiment, the OPS filter 110 may be coated and/or treated with antimicrobial agents to prevent microbial growth and contamination. The antimicrobial agents used for coating may include, but are not limited to, silver nanoparticles, essential oils, and natural antimicrobial peptides. In another embodiment, the OPS filter 110 may be equipped with a sensor system within the feeding bottle. The sensor system may monitor the performance of the OPS filter 110. Further, the sensor system may detect and report the presence of microplastics or other contaminants in real time. The report obtained from the sensor may be communicated to a mobile application, and/or a digital display on the bottle, providing users with immediate feedback on the milk quality.

In another embodiment, the OPS filter 110 may be a replaceable cartridge within the feeding bottle. The replaceable cartridge may facilitate users to easily replace the OPS filter 110 after a certain period and usage. Further, the replaceable cartridge may ensure that the filter remains effective over time. In an embodiment, the replaceable cartridge may include a locking mechanism to ensure secure placement within the bottle.

In an embodiment, the OPS filter 110 may be installed across various container shapes and sizes. The OPS filter 110 may have customizable components, such as adjustable attachments and flexible materials, to conform to different bottleneck sizes and shapes. The customizable components may be tailored to securely fit bottles with varying neck dimensions, ensuring a snug and leak-proof installation. Further, the customizable components of the OPS filter 110 may allow adjustment to bottles with curved or irregular interiors, maintaining effectiveness in diverse configurations. In an embodiment, the OPS filter 110 may include universal fit options and user-friendly installation mechanisms to enhance versatility. Interchangeable OPS filter 110 and adjustable sealing mechanisms provide users with the ability to customize the filter according to specific bottle requirements, while also facilitating easy placement and removal. The combination of modular design, adaptability, and ease of use may ensure that the OPS filter 110 may be seamlessly integrated into a wide range of feeding bottles and containers, offering a reliable solution for improving milk quality in various applications.

In an embodiment, the OPS filter 110 may exhibit an adsorption efficiency ranging between 75% to 81.2% at a pH of 6, providing optimal performance for filtering contaminants without significantly altering the milk's nutritional composition. The OPS filter 110 may possess mechanical properties such as a compressive strength of 176 kPa and a strain capacity of 66%, ensuring durability and resilience under repetitive use. Further, the OPS filter 110 hydrogel matrix may be easily removable for cleaning and maintenance, ensuring sustained filtration efficacy over multiple uses.

In an embodiment, the OPS filter 110 installed within the feeding bottle 102 may ensure that the milk is purified at the point of consumption. As milk flows from the container 104 through the OPS filter 110 and subsequently through the rubber nipple 108, microplastics are trapped within the filter matrix, enhancing the safety and quality of the milk provided to the infant.

In further embodiments, the feeding bottle 102 may be compatible with various sizes and shapes of OPS filters 110, allowing customization based on specific filtration needs or milk composition. The materials used for the container 104, cap 106, and rubber nipple 108 may also be adapted to enhance compatibility with the OPS filter 110, ensuring smooth integration and optimal filtration performance during feeding.

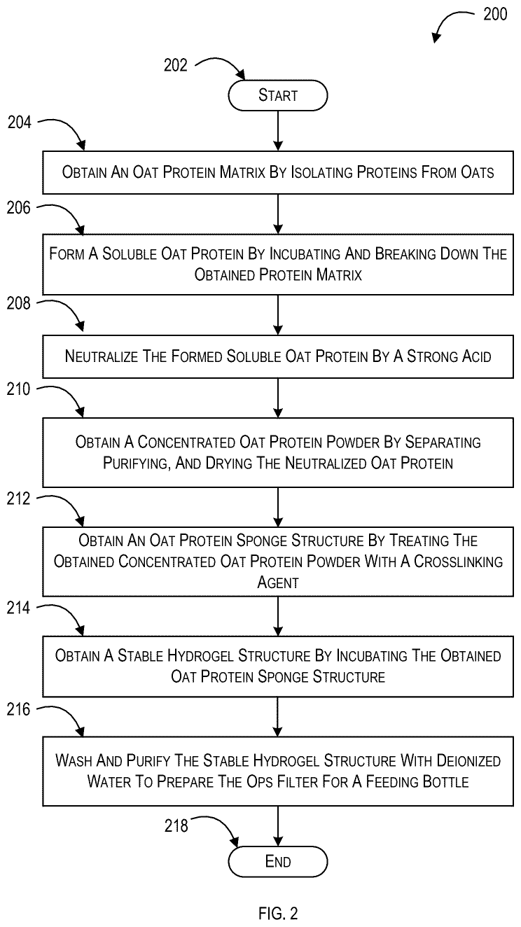

FIG. 2 illustrates a flowchart 200 of method for the preparation of the Oat Protein Sponge (OPS) filter for feeding bottles, in accordance with an embodiment of the present disclosure. The method begins at step 202.

At step 204, the method may include obtaining an oat protein matrix by isolating proteins from oats by cell disruption using an alkali. The oats may be cultivated through tissue culture and/or hydroponics. The alkali may include Sodium Hydroxide (NaOH) or Potassium Hydroxide (KOH). In an embodiment, the oat protein matrix may be enhanced with additional functional additives during preparation. The additives may include natural polymers and stabilizers to improve the mechanical properties of the OPS filter and the filtration efficiency of the OPS filter. Further, the additives may be added at various stages, such as during the crosslinking step, to modify the filtering, flexibility, porosity, and adsorption capacity. The additives may include, but are not limited to, guar gum, alginate, and chitosan.

At step 206, the method may include forming a soluble oat protein by incubating and breaking down the obtained protein matrix. The incubation may facilitate the protein matrix to undergo controlled degradation, to make the proteins more soluble. The breakdown of the protein matrix may convert the solid protein structures into a soluble form.

At step 208, the method includes neutralizing the formed soluble oat protein by a strong acid. The strong acids may include Hydrochloric Acid (HCl) and/or Sulfuric Acid (H₂SO₄). The neutralization may bring the solution to a neutral pH. The neutral pH may ensure that the oat protein is in a stable and balanced state

At step 210, the method may include obtaining a concentrated oat protein by separating, purifying, and drying the neutralized oat protein. In an embodiment, the neutralized oat protein may undergo separation to obtain the purified protein by removing any impurities or unwanted components. The purified oat protein may be dried to eliminate any residual moisture to obtain concentrated oat protein.

At step 212, the method may include obtaining an oat protein sponge structure by treating the obtained concentrated oat protein with a crosslinking agent. The crosslinking agent may include epichlorohydrin. In an embodiment, treating the obtained concentrated oat protein may include adding a predefined amount of the crosslinking agent to the obtained concentrated oat protein. The predefined amount may be 600 μL. Further, treating the obtained concentrated oat protein may include stirring for a predefined time interval to obtain the oat protein sponge structure. The predefined time interval is 2 hours.

At step 214, the method may include obtaining a stable hydrogel structure by incubating the obtained oat protein sponge structure. The incubation process may be performed for a time interval ranging from 8 to 12 hours. Further, the incubation may facilitate the structure to form a stable hydrogel with a porous texture.

At step 216, the method may include washing and purifying the stable hydrogel structure with deionized water to prepare the OPS filter for a feeding bottle. The washing and purifying may make the OPS filter safe for use and remove any residual chemicals, such as excess crosslinking agents or by-products. The OPS filter may filter out microplastics from milk stored in the feeding bottles during feeding sessions. Further, the OPS filter may have a pore size range from 0.49 to 1.54 μm to remove the microplastics from the milk and allow passage of nutrients. Furthermore, the OPS filter adsorption efficiency may range from 75% to 81.2% at a pH of 6. The method ends at step 218.

In an embodiment, the OPS filter may be prepared from oat-derived proteins, and processed through a series of steps including crosslinking and hydrogel formation, to create a sponge-like structure with defined filtration capabilities. Oat protein extraction may be performed by cultivating high-quality oat plants. The oat plants may be cultivated using tissue culture or hydroponics to ensure optimal protein yield. Tissue culture may include growing oat cells in a controlled laboratory setting, where nutrients and environmental factors are precisely regulated.

In an embodiment, the oat protein may be obtained by hydroponics which may allow for the growth of oats in a water-based, nutrient-rich solution without soil, promoting faster growth and a higher protein content. Once harvested, the oat plants may undergo processing, during which an alkali solution, such as sodium hydroxide or potassium hydroxide, may be applied. The alkaline treatment may disrupt the cell walls of the oats, enabling the release of soluble proteins. The harvested oats undergo processing, employing alkali solutions such as sodium hydroxide (NaOH) or potassium hydroxide (KOH) to disrupt the oat cells, releasing proteins. The alkaline treatment may effectively break down the cell walls and facilitate the extraction of soluble proteins. The soluble protein may be utilized in forming the Oat Protein Sponge (OPS). The soluble oat protein solution may undergo neutralization with a strong acid, such as hydrochloric acid (HCl), to adjust the pH to a neutral level.

In an embodiment, the neutralized oat protein may undergo separation to obtain the purified protein by removing any impurities or unwanted components. The purified oat protein may be dried to eliminate any residual moisture to obtain concentrated oat protein.

In an embodiment, concentrated protein powder may undergo crosslinking to obtain the sponge-like structure of the OPS filter. The crosslinking may include the chemical bonding of protein molecules, resulting in a stable network. Epichlorohydrin may serve as the crosslinking agent, reacting with the amine groups present in the oat proteins and forming covalent bonds between protein molecules. The covalent bonds may create a robust, interconnected protein network, establishing a three-dimensional matrix that serves as the foundation for the sponge-like structure.

In an embodiment, incubating the crosslinked oat protein matrix may facilitate hydrogel formation. During the incubation, the cross linked protein network may absorb water and swell into a gel-like material characterized by interconnected pores. The hydrogel may demonstrate a high-water content and a porous, sponge-like structure, with precise pore sizes essential for effectively filtering microplastics and other contaminants.

In an embodiment, the obtained hydrogel may undergo extensive washing and purification to remove residual chemicals, such as excess crosslinking agents or by-products. The purification process may ensure that the final OPS filter remains non-toxic and safe for use in a feeding bottle. Once purified, the hydrogel may undergo a drying process to eliminate water content while maintaining the porous structure of the sponge. The resulting dried sponge may retain the porous network with pore sizes.

In an embodiment, the OPS filter may have a pore size ranging from approximately 0.49 μm to 1.54 μm, allowing it to effectively capture microplastics present in the milk while egressing the passage of essential nutrients.

The disclosed Oat Protein Sponge (OPS) filter for feeding bottles. The OPS filter may filter microplastics from the stored milk before the milk egresses during the feeding session. The natural oat protein utilized in the OPS filter may offer both environmental and health benefits. By leveraging the inherent adsorption properties of oat proteins, the OPS filter avoids the need for synthetic chemicals, reducing potential adverse interactions with milk. Additionally, the biodegradable nature of oat proteins aligns with sustainable practices, minimizing the environmental impact and contributing to an eco-friendly solution for feeding bottle filtration.

The OPS filter may offer a significant reduction in environmental impact due to its use of biodegradable materials. The oat proteins utilized in the filter are derived from natural sources, which decompose efficiently in environmental conditions, minimizing long-term waste and pollution. The preparation of the OPS filter avoids harmful chemicals and leverages sustainable practices, such as using renewable plant-based materials. Biocompatibility of the OPS filter ensures that no toxic substances or adverse reactions are introduced when in contact with milk or used in feeding bottles. The OPS filter maintains the safety and integrity of the milk while simultaneously promoting environmental sustainability. The biodegradability and non-toxic nature of the OPS filter supports an eco-friendlier approach to product design, aligning with contemporary environmental and health standards.

The manufacturing process of the OPS filter minimizes carbon emissions and resource consumption. Using oat proteins derived from renewable agricultural sources supports sustainable agricultural practices and reduces dependence on non-renewable materials. The crosslinking agents and processing methods employed are chosen for their lower environmental impact compared to traditional chemical alternatives. The efficient utilization of resources throughout the filter's life cycle i.e. from production to disposal contributes to a reduced overall carbon footprint. This environmentally conscious approach not only addresses current ecological concerns but also enhances the appeal of the OPS filter to environmentally aware consumers and manufacturers seeking to adopt greener technologies.

The production process utilizing oat proteins and natural crosslinking agents, to prepare the OPS filter, is cost-effective compared to conventional filtration technologies. Utilizing readily available and biodegradable materials reduces material costs and waste. Additionally, the streamlined manufacturing process encompassing protein extraction, crosslinking, and hydrogel formation contributes to reduced production costs and improved scalability, making the OPS filter an economically viable solution for large-scale applications.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

While the foregoing describes various embodiments of the invention, other and further embodiments of the invention may be devised without departing from the basic scope thereof. The scope of the invention is determined by the claims that follow. The invention is not limited to the described embodiments, versions, or examples, which are included to enable a person having ordinary skill in the art to make and use the invention when combined with information and knowledge available to the person having ordinary skill in the art.

I/We claim:

1. A method of preparation of Oat Protein Sponge (OPS) filter for a feeding bottle, the method comprising:
obtaining an oat protein matrix by isolating proteins from oats by cell disruption using an alkali;
forming a soluble oat protein by incubating and breaking down the obtained protein matrix;
neutralizing the formed soluble oat protein by a strong acid;
obtaining a concentrated oat protein by at least one of: separating purifying, and drying the neutralized oat protein;
obtaining an oat protein sponge structure by treating the obtained concentrated oat protein with a crosslinking agent;
obtaining a stable hydrogel structure by incubating the obtained oat protein sponge structure; and
washing and purifying the stable hydrogel structure with deionized water to prepare the OPS filter for a feeding bottle.

2. The method as claimed in claim 1, wherein the oats are cultivated through at least one of: tissue culture and hydroponics.

3. The method as claimed in claim 1, wherein the alkali includes at least one of: Sodium Hydroxide (NaOH) and Potassium Hydroxide (KOH).

4. The method as claimed in claim 1, wherein the strong acid includes at least one of: Hydrochloric acid (HCl) and Sulfuric acid (H₂SO₄).

5. The method as claimed in claim 1, wherein the crosslinking agent includes at least epichlorohydrin, and treating the obtained concentrated oat protein includes the steps of:
adding a predefined amount of the crosslinking agent to the obtained concentrated oat protein; and
stirring for a predefined time interval to obtain the oat protein sponge structure.

6. The method as claimed in claim 5,
wherein the predefined amount is 600 μL and the predefined time interval is 2 hours.

7. The method as claimed in claim 1, wherein the OPS filter filters out microplastics from milk stored in the feeding bottles during feeding sessions.

8. The method as claimed in claim 1,
wherein the OPS filter has a pore size range from 0.49 to 1.54 μm to remove the microplastics from the milk and allow passage of nutrients;
wherein the incubation of the obtained oat protein sponge structure to obtain the stable hydrogel structure is performed for a time interval ranging from 8 to 12 hours;
wherein an adsorption efficiency of the OPS filter ranges from 75% to 81.2% at a pH of 6; and
wherein the OPS filter has a compressive strength of 176 kPa and a strain capacity of 66%.

9. A feeding bottle with an Oat Protein Sponge (OPS) filter, the feeding bottle comprising:
a container to store milk;
a cap to be placed on the container to prevent the stored milk from spilling and contamination;
a rubber nipple coupled to the cap for egressing of the milk during feeding sessions; and
an Oat Protein Filter (OPS) installed in the feeding bottle, such that the OPS filters microplastics from the stored milk before the milk egresses from the rubber nipple during the feeding sessions, wherein the OPS is formed by the steps of:
obtaining an oat protein matrix by isolating proteins from oats by cell disruption using an alkali;
forming a soluble oat protein by incubating and breaking down the obtained protein matrix;
neutralizing the formed soluble oat protein by a strong acid;
obtaining a concentrated oat protein by at least one of: separating purifying, and drying the neutralized oat protein;
obtaining an oat protein sponge structure by treating the obtained concentrated oat protein with a crosslinking agent;
obtaining a stable hydrogel structure by incubating the obtained oat protein sponge structure; and
washing and purifying the stable hydrogel structure with deionized water to prepare the OPS filter for a feeding bottle.

10. The feeding bottle as claimed in claim 9, wherein at least one of: the container and the cap is made up of a material selected from a group of materials including at least one of: metal, plastic, fibre, and silicon.
